(19) United States
(12) Patent Application Publication
(10) Pub. No.: US 2022/0339014 A1
(43) Pub. Date: Oct. 27, 2022

(54) MECHANICAL DETACHMENT SYSTEM WITH A HOLD-RELEASE STRUCTURE FOR DEPLOYMENT OF ENDOVASCULAR DEVICES

(71) Applicant: DeepIn Technologies, LLC, San Jose, CA (US)

(72) Inventors: Ross Soltanian, San Jose, CA (US); Gregory M. Mast, Morgan Hill, CA (US)

(21) Appl. No.: 17/722,166

(22) Filed: Apr. 15, 2022

Related U.S. Application Data
(60) Provisional application No. 63/179,163, filed on Apr. 23, 2021.
(60) Provisional application No. 63/183,539, filed on May 3, 2021.

(51) Int. Cl. A61F 2/966; A61B 17/12

(52) U.S. Cl. CPC A61F 2/966; A61B 17/12113; A61B 17/1214; A61F 2002/9665; A61F 2210/0014; A61B 2017/12054; A61B 2017/00867

(57) ABSTRACT

A delivery system employs a hold-release structure to deploy an implant at a target site in the vasculature of a patient. The hold-release structure may include two or more grasping members configured to close and exert an inward clamping force to hold the implant when the grasping members are constrained in a tubular member. The grasping members can open when unconstrained allowing the implant to be released. Alternatively, the hold-release structure may include two or more radially expandable members configured to exert an outward radial force when constrained by the tubular member allowing the hold-release structure to hold the implant against the tubular member. The radially expandable members can be configured to create a friction force on the implant allowing the hold-release structure to move the implant relative to the tubular member.

### CROSS-REFERENCE TO RELATED APPLICATIONS

This application claims priority to U.S. provisional patent application No. 63/179,163 filed Apr. 23, 2021 entitled “Mechanical Detachment System for Deployment of Endovascular Devices,” and 63/183,539 filed May 3, 2021 entitled “Mechanical Detachment System for Deployment of Endovascular Devices (Conforming Expander),” the disclosures of all of which are hereby incorporated by reference in their entirety.

### TECHNICAL FIELD

This application relates generally to medical devices and methods. In particular, various embodiments of endovascular systems and mechanical detachment systems for deploying implants within the vasculature of a human body are described.

### BACKGROUND

Implants such as embolic devices are known in treatment of vascular disorders such as aneurysms and in peripheral thrombectomy. An aneurysm is a bulge or swelling formed on a wall of an artery in the brain or other locations of a human body. A brain aneurysm can cause severe pain, and if ruptured, lead to fetal stroke. In a non-invasive or minimally invasive treatment of aneurysms, an embolic device such as a coil, stent, or intrasaccular web may be placed in or at the aneurysm to isolate the aneurysm from blood flow, and/or, promote thrombus formation at the site. The placement of an embolic device is typically accomplished using a delivery system, which steers the embolic device through the vasculature of the patient to the location of the aneurysm. Once positioned at or in the aneurysm, the embolic device is detached from the delivery system by applying thermal or electrolytic power or by activating a mechanical detachment mechanism.

Conventional systems or methods for delivering and deploying embolic devices often present risks of prematurely or inadvertently releasing the embolic devices before deployment at the target location. For example, conventional systems reply on some sort of mechanical coupling to join an implant to a delivery wire. Such systems have limitations when navigating through vascular paths which, quite frequently, cause premature detachment during deployment or retraction. This is especially the case in treating brain aneurysms during which a delivery system would have to navigate through a tortuous vascular path, where advancement and retraction of the delivery system is often required in order to accurately place the embolic device to reduce errors that may result in significant damage to the brain.

Therefore, there remains a general need for improved systems and methods of delivering implants for treating vascular disorders. It would be desirable to provide a delivery system that can reliably and controllably navigate through the vasculature of a human body in delivering implants and reduce the risks of premature or inadvertent release of the implants before deployment at a target site.

### SUMMARY

In one aspect, embodiments of the disclosure feature a system for delivering an implant in a patient. In general, an embodiment of the delivery system comprises a tubular member having a lumen, a delivery wire having a proximal end portion and a distal end portion extending in the lumen of the tubular member, and a hold-release structure coupled to the distal end portion of the delivery wire. The hold-release structure comprises two or more grasping members and is slidably movable in the lumen of the tubular member between a proximal first position and a distal second position. In the proximal first position, the two or more grasping members are constrained by the tubular member allowing the two or more grasping members to close and exert an inward clamping force to hold the implant. In the distal second position, the two or more grasping members are unconstrained allowing the two or more grasping members to open to release the implant.

In various embodiments of the aspect, the two or more grasping members comprise an inward step configured to contact an outer surface of the implant in exerting the clamping force.

In various embodiments of the aspect, the hold-release structure is constructed from a material comprising a shape-memory material, and the two or more grasping members have a pre-determined open configuration when unconstrained.

In various embodiments of the aspect, the hold-release structure comprises a tubular body and the two or more grasping members when constrained constitute an extension of the tubular body.

In another aspect, embodiments of the disclosure feature an endovascular system. In general, an embodiment of the endovascular system comprises an implant and a delivery device operable to deploy the implant at a target site in a vasculature of a patient. The delivery device comprises a tubular member having a lumen, a delivery wire having a proximal end portion and a distal end portion extending in the lumen of the tubular member, and a hold-release structure coupled to the distal end portion of the delivery wire. The hold-release structure comprises two or more grasping members and is slidably movable in the lumen of the tubular member between a proximal first position and a distal second position. In the proximal first position, the two or more grasping members are constrained by the tubular member allowing the two or more grasping members to close and exert an inward clamping force to hold the implant. In the distal second position, the two or more grasping members are unconstrained allowing the two or more grasping members to open to release the implant.

In various embodiments of the aspect, the implant comprises an embolic coil, a stent, or an intrasaccular web. In a specific embodiment, the implant comprises a stent.

In various embodiments of the aspect, the two or more grasping members of the hold-release structure comprise an inward step configured to contact an outer surface of the implant in exerting the clamping force.

In various embodiments of the aspect, the hold-release structure is constructed from a material comprising a shape-memory material, and the two or more grasping members have a pre-determined open configuration when unconstrained.

In various embodiments of the aspect, the hold-release structure comprises a tubular body and the two or more grasping members when constrained constitute an extension of the tubular body.

In a further aspect, embodiments of the disclosure feature a system for delivering an implant in a patient. In general, an embodiment of the delivery system comprises a tubular member having a lumen, a delivery wire having a proximal end portion and a distal end portion extending in the lumen of the tubular member, and a hold-release structure coupled to the distal end portion of the delivery wire. The hold-release structure comprises two or more radially expandable members and is slidably movable in the lumen of the tubular member between a proximal first position and a distal second position. The two or more radially expandable members are configured to exert an outward radial force when constrained by the tubular member allowing the hold-release structure to hold an implant against the tubular member in the proximal first position. The two or more radially expandable members are configured to create a friction force on the implant allowing the hold-release structure to move the implant relative to the tubular member from the proximal first position to the distal second position.

In various embodiments of the aspect, the two or more radially expandable members are configured to exert the outward radial force to an inner surface of the implant.

In various embodiments of the aspect, the hold-release structure is constructed from a shape-memory material forming the two or more radially expandable members in a pre-determined open configuration when unconstrained.

In various embodiments of the aspect, the two or more radially expandable members comprise a contact surface having a shape generally conforming to the inner surface of the implant.

In various embodiments of the aspect, the two or more radially expandable members comprise a coating or pad configured to provide an increased friction force between the two or more radially expandable members and the implant.

In a further aspect, embodiments of the disclosure feature an endovascular system. In general, an embodiment of the endovascular system comprises an implant and a delivery device operable to deploy the implant at a target site in a vasculature of a patient. The delivery device comprises a tubular member having a lumen, a delivery wire having a proximal end portion and a distal end portion extending in the lumen of the tubular member, and a hold-release structure coupled to the distal end portion of the delivery wire. The hold-release structure comprises two or more radially expandable members and is slidably movable in the lumen of the tubular member between a proximal first position and a distal second position. The two or more radially expandable members are configured to exert an outward radial force when constrained by the tubular member allowing the hold-release structure to hold an implant against the tubular member in the proximal first position. The two or more radially expandable members are configured to create a friction force on the implant allowing the hold-release structure to move the implant relative to the tubular member from the proximal first position to the distal second position.

In various embodiments of the aspect, the implant comprises an embolic coil, a stent, or an intrasaccular web. In a specific embodiment, the implant comprises a stent.

In various embodiments of the aspect, the two or more radially expandable members are configured to exert the outward radial force to an inner surface of the stent.

In various embodiments of the aspect, the hold-release structure is constructed from a shape-memory material forming the two or more radially expandable members in a pre-determined open configuration when unconstrained.

In various embodiments of the aspect, the two or more radially expandable members comprise a contact surface having a shape generally conforming to the inner surface of the implant.

In various embodiments of the aspect, the two or more radially expandable members comprise a coating or pad configured to provide an increased friction force between the two or more radially expandable members and the implant.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1A depicts an example of an endovascular system including an embolic device and a delivery device in a delivery configuration according to an embodiment of the disclosure.

FIG. 1B depicts the example endovascular system shown in FIG. 1A in a deployed configuration according to an embodiment of the disclosure.

FIG. 1C depicts the example endovascular system shown in FIG. 1A in a partially deployed configuration according to an embodiment of the disclosure.

FIG. 2A depicts an example of a hold-release structure in a constrained state according to an embodiment of the disclosure.

FIG. 2B depicts the example hold-release structure in an unconstrained state according to an embodiment of the disclosure.

FIG. 3A depicts an example of an endovascular system including an embolic device and a delivery device in a delivery configuration according to another embodiment of the disclosure.

FIG. 3B depicts the example endovascular system shown in FIG. 3A in a deployed configuration according to an embodiment of the disclosure.

FIG. 3C depicts the example endovascular system shown in FIG. 3A in a partially deployed configuration according to an embodiment of the disclosure.

FIG. 4A depicts an example of a hold-release structure in a constrained state according to an embodiment of the disclosure.

FIG. 4B depicts the example hold-release structure in an unconstrained state according to an embodiment of the disclosure.

### DETAILED DESCRIPTION OF EMBODIMENTS

With reference to the figures, various embodiments of an endovascular system and a detachment system for delivering and deploying implants will now be described. It should be noted that the figures are intended for illustration of embodiments but not for exhaustive description or limitation on the scope of the disclosure. Alternative structures and components will be readily recognized as being viable without departing from the principle of the claimed invention.

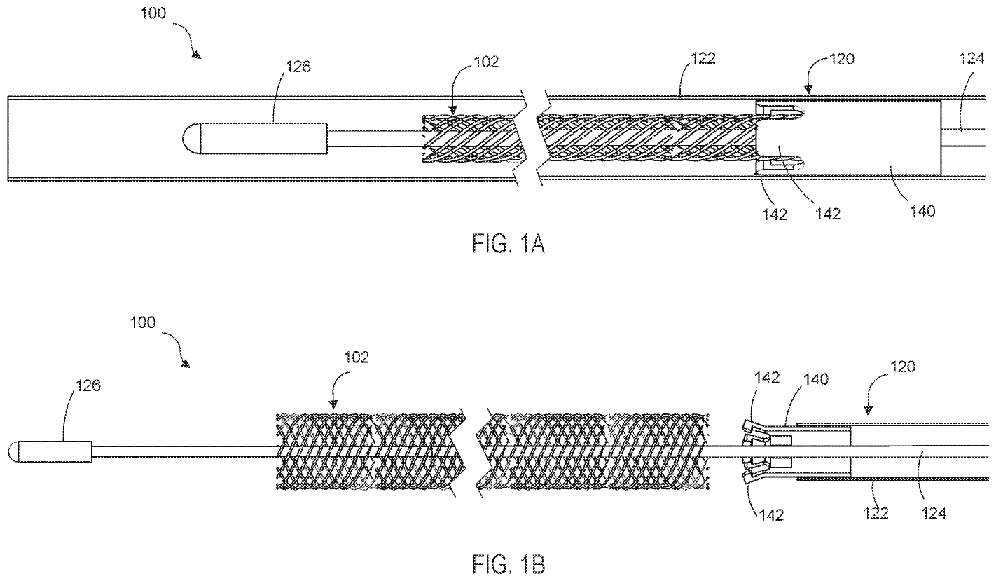

FIGS. 1A-1C illustrate an example of an endovascular system 100 according to some embodiments of the disclosure (GRASPER). In a broad overview, the example endovascular system 100 includes an implant 102 and a delivery device 120 operable to deliver and deploy the implant 102 at a target site in a patient. The delivery device 120 generally includes an elongate tubular member 122 and an elongate delivery wire 124. A hold-release structure 140 is coupled to the delivery wire 124, holding the implant 102 during delivery and releasing the implant 102 during deployment. The hold-release structure 140 comprises two or more grasping members 142. In a delivery state shown in FIG. 1A, the two or more grasping members 142 are constrained by the tubular member 122, allowing the two or more grasping members 142 to close and exert an inward clamping force to a proximal end portion of the implant 102 to hold the implant. In a release state shown in FIG. 1B, the two or more grasping members 142 exit the tubular member 122 and are unconstrained. As such, the two or more grasping members 142 may open or recoil to their pre-determined configuration, allowing the implant 102 to be released or deployed at the target site. If repositioning of the endovascular system 100 is needed for accurate deployment, the implant 102 can be re-sheathed or recaptured into the tubular member 122 before the implant 102 is completely released, by e.g., proximally pulling the delivery wire 124, as shown in FIG. 1C.

With reference to FIGS. 1A-1C, the elongate tubular member 122 can be a sheath, microcatheter, or any other suitable tubular member defining a lumen. The elongate tubular member 122 may include a proximal end portion which may remain outside of the patient and is accessible to the user or physician when the endovascular system 100 is in use. The distal end portion of the tubular member 122 may be sized and dimensioned to reach a remote location in the vasculature of the patient, such as in a blood vessel adjacent to an aneurysm neck, a bifurcated blood vessel, an occlusion in a blood vessel, or the like. The hold-release structure 140 may be disposed at the distal end portion of the tubular member 122. While not shown, the elongate tubular member 122 may include one or more sections or regions each of which may have different configurations and/or characteristics. For example, the distal end portion of the elongate tubular member 122 may include a flexible section or region constructed e.g., of a coil to provide proper bending or deflection. A flexible distal end portion would allow the endovascular system 100 to navigate more easily through tortuous regions of the vasculature to remote locations in the patient. The proximal end portion may be constructed of a stiffer material e.g., of a rigid metal hypertube, to provide structural stability and sufficient pushability. In general, the elongate tubular member 122 or a section of the elongate tubular member 122 may be constructed from suitable metals such as stainless steel, nickel, titanium, nitinol, alloys of metals, biocompatible polymers, shape memory polymers, or combinations thereof. The distal end portion of the elongate tubular member 122 may have an outer diameter less than the outer diameter of the proximal end portion to reduce the profile of the distal end portion and facilitate navigation through tortuous vasculature. While not shown, the elongate tubular member 122 may include one or more markers which can be viewed e.g., via fluoroscopy, to assist the physician in operation of the endovascular system 100.

With reference to FIGS. 1A-1C, the elongate delivery wire 124 has a proximal end portion and a distal end portion. The proximal end portion of the delivery wire 124 may remain outside of the patient and is accessible to the user or physician when the endovascular system 100 is in use. The distal end portion of the delivery wire 124 couples the hold-release structure 140. The delivery wire 124 may include a distal tip 126 shaped or configured to make the delivery wire more flexible or atraumatic. While not shown, the distal end portion of the delivery wire 124 may include a section or region constructed e.g., of a coil to provide proper bending or deflection. One or more markers may also be coupled to the delivery wire 124 to assist the physician to operate the endovascular system 100 via fluoroscopy. The delivery wire 124 may be constructed from suitable metals such as stainless steel, nickel, titanium, nitinol, alloys of metals, biocompatible polymers, shape memory polymers, or combinations thereof.

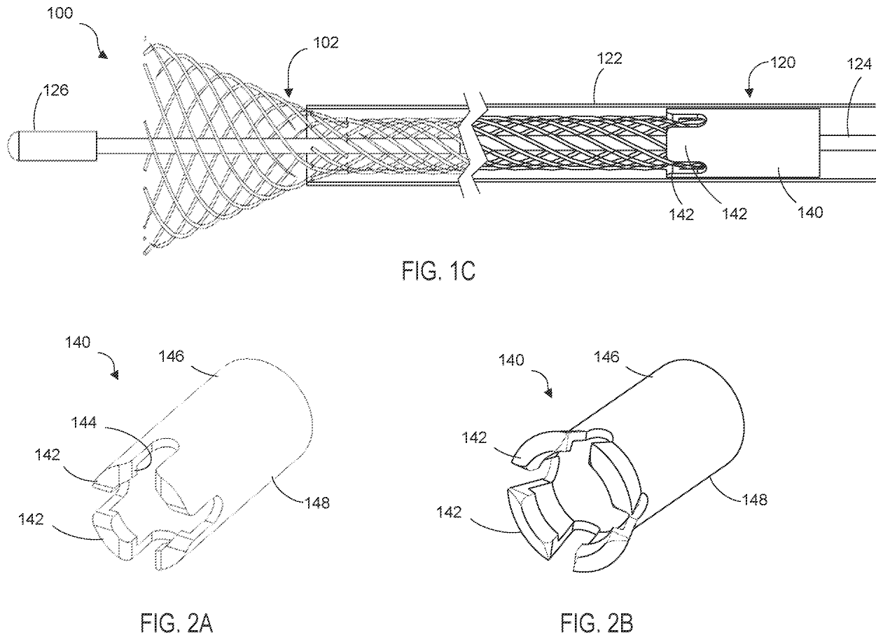

With reference to FIGS. 1A-1C and 2A-2B, the hold-release structure 140 may be coupled to the distal end portion of the delivery wire 124. The hold-release structure 140 can serve to hold or secure the implant 102 during delivery to the target site, re-sheath or recapture the implant 102 into the tubular member 122 for repositioning, and release or deploy the implant 102 to the target site.

With reference to FIGS. 1A-1C and 2A-2B, the hold-release structure 140 may be sized and/or shaped to be disposed within the tubular member 122. The hold-release structure is slidably movable in the lumen of the tubular member 122. By way of example, the hold-release structure 140 may be moved distally relative to the tubular member 122 by pushing the delivery wire 124, and/or proximally relative to the tubular member 122 by pulling the delivery wire 124. Alternatively, the hold-release structure 140 may be distally and/or proximally moved relative to the tubular member 122 by pulling and/or pushing the tubular member 122. The hold-release structure 140 may be fixedly coupled to the delivery wire 124 by any suitable means such as via soldering, welding, adhesive bonding, etc.

With reference to FIGS. 1A-1C and 2A-2B, the hold-release structure 140 may comprise two or more grasping members 142. The two or more grasping members 142 may have a closed state (FIG. 2A) when constrained inside the tubular member 122 and an open state (FIG. 2B) when unconstrained. The two or more grasping members 142 may include a step 144 or other features for grasping the implant 102. In a preferred embodiment, the two or more grasping members 142 may be biased or formed such that the grasping members 142 have a predetermined open configuration when unconstrained or in a natural state. When constrained or compressed, the two or more grasping members 142 may close and exert an inward clamping force. By way of example, when constrained inside the tubular member 122 during delivery, the two or more grasping members 142 close and exert an inward clamping force to hold the implant 102 (FIGS. 1A and 1C). When unconstrained outside the tubular member 122 in a release state, the two or more grasping members 142 recoil to their natural open state, allowing the implant 102 to be released (FIG. 1B). As used herein, the term “inward” or its grammatical equivalent refers to a direction or an orientation that is towards the delivery wire 124.

With reference to FIGS. 2A-2B, the hold-release structure 140 can be constructed from a shape-memory material. Suitable shape-memory materials include nickel-titanium alloy Nitinol and other metal alloys. By way of example, the two or more grasping members 142 may be formed by cutting a tubular wall 146 of a shape-memory material, allowing the grasping members 142 to be biased such that they have a predetermined open configuration when in a natural state or unconstrained. Any suitable techniques may be used to cut the tubular wall 260, including laser cutting, etching, etc. known in the art.

With reference to FIGS. 2A-2B, in a preferred embodiment, the hold-release structure 140 comprises a proximal portion including a tubular body 148 and distal portion including two or more grasping members 142 extended from the tubular body 148. The tubular body 148 may have an outer diameter slightly smaller than the inner diameter of the elongate tubular member 122. The lumen defined by the tubular body 148 may have a size and/shape to receive the delivery wire 126. The tubular body 148 may be fixedly coupled to the delivery wire 124 by soldering, welding, adhesive bonding, etc. The two or more grasping members 142 may be formed such that when constrained the grasping members 142 constitute an extension of the tubular body 148.

With reference to FIGS. 1A-1C, the implant 102 can be any suitable implantable device compatible with the delivery system 100 of the disclosure. By way of example, the implant 102 can be an embolic device such as a stent, coil, or intrasaccular web used for treatment of brain aneurysms and/or peripheral thrombectomy. The implant 102 can also be a dilation device, filter, thrombectomy device, atherectomy device, flow restoration device, etc. for treatment of other disorders in other locations of a human body. For illustration purpose, the implant 102 is shown in FIGS. 1A-1C as an expandable stent. It should be noted that the scope of the disclosure and appended claims are not limited to a specific type of an implant, and the hold-release structure 140 disclosed herein can be used with any other suitable implants.

In operation, the implant 102 may be pre-loaded with the hold-release structure 140 inside a delivery sheath. The implant 102 and the hold-release structure 140 can be then transferred to a microcatheter to be delivered and deployed at a target site for treatment of a disorder within the vasculature of a patient. In an embodiment for treating neurovascular conditions such as aneurysm or for peripheral thrombectomy, a microcatheter may be introduced to the target site through an access e.g., in the femoral artery or groin area of the patient by using an introducer sheath or guiding catheter. The microcatheter may be guided to the target site through the use of a guidewire. The guidewire is visible via fluoroscopy, allowing the microcatheter to be reliably advanced over the guidewire to the target site.

Once the target site has been accessed with the microcatheter tip, the guidewire can be withdrawn, clearing the lumen of the microcatheter. The endovascular system 100 including the implant 102 and the delivery device 120 in a delivery configuration, can be placed into the proximal open end of the microcatheter and advanced through the microcatheter. When the implant 102 reaches the distal end of the microcatheter, it can be deployed from the microcatheter and positioned at the target site. The physician may advance and retract the implant 102 several times to obtain a desirable position of the implant within the vasculature. Once the implant 102 is satisfactorily positioned, the physician may push the delivery wire 124 distally, allowing the implant 102 to exit the delivery device, thereby releasing the implant at the target site. The elongate tubular member 122 can be then removed from the microcatheter, and the microcatheter can be withdrawn from the vasculature of the patient.

Various embodiments of an endovascular system and a system for deploying implants within a human body have been described. Advantageously, the delivery system of the disclosure can enhance securement of the implant during delivery. The enhanced securement of the delivery system significantly reduces the risks of inadvertent or premature release of the implant when the delivery system is advanced or retracted in navigating through a tortuous vascular path in the human body. Conventional delivery systems rely on coupling of an implant to the delivery wire. When navigating through tortuous paths, the implant ovalizes, causing pre-mature detachment of the implant from the delivery wire. Conventional delivery systems also rely on the inner diameter dimension of the microcatheter. A minor variation in the inner diameter dimension would result in pre-mature deployment. The hold-release structure of the disclosure uses grasping members to hold or grasp the implant from the outside of the implant. As such, the implant has the capability of compensating for the variation in microcatheter's inner diameter, thereby reducing the risks of pre-mature detachment when navigating through tortuous paths.

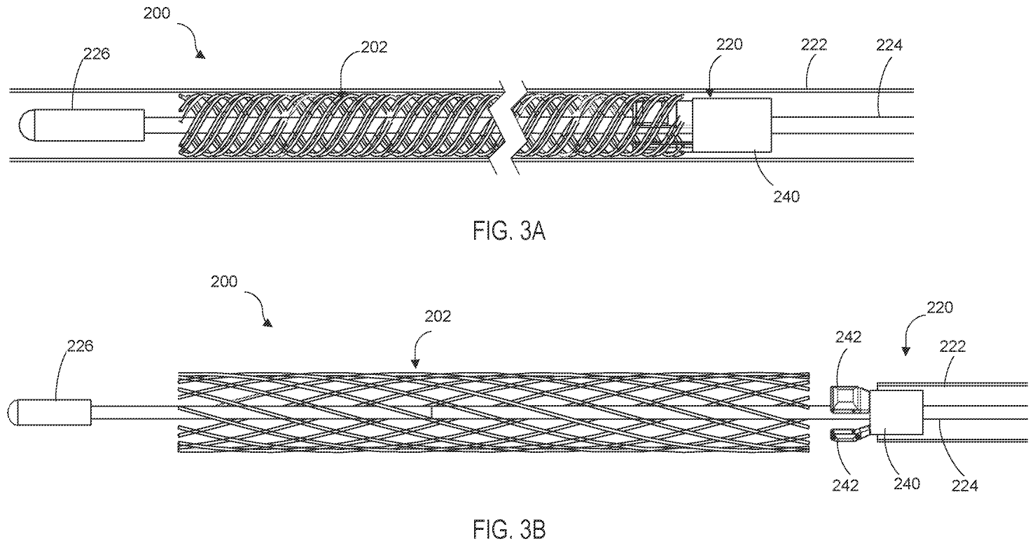

FIGS. 3A-3C illustrate an example of an endovascular system 200 according to some embodiments of the disclosure (CONFORMING EXPANDER). In a broad overview, the example endovascular system 200 includes an implant 202 and a delivery device 220 operable to deliver and deploy the implant 202 at a target site in a patient. The delivery device 220 generally includes an elongate tubular member 222 and an elongate delivery wire 224. A hold-release structure 240 is coupled to the delivery wire 224, holding the implant 202 during delivery and releasing the implant 202 during deployment. The hold-release structure 240 can be disposed within the implant 202, which in turn can be disposed within the tubular member 222. The hold-release structure 240 comprises two or more radially expandable members 242. In a delivery state shown in FIG. 3A, the two or more radially expandable members 242 are constrained by the tubular member 222, allowing the radially expandable members 242 to exert an outward radial force to the inner surface of the implant 202, thereby holding the implant 202 against the tubular member 222. To release the implant 202 as shown in FIG. 3B, the hold-release structure 240 can be distally moved e.g., by pushing the delivery wire 224. Due to the friction force between the implant 202 and the radially expandable members 242, the implant 202 can be distally moved with the hold-release structure 240 when pushed by the delivery wire 224. As the two or more expandable members 242 exit the tubular member 222 shown in FIG. 3B, the implant 202 can be released and deployed at the target site. If repositioning of the endovascular system 200 is needed for accurate deployment, the partially released implant 202 can be re-sheathed or recaptured into the tubular member 222 before the implant 202 is completely released, by e.g., proximally pulling the delivery wire 224 and the hold-release structure 240, as shown in FIG. 3C. Because the radially expandable members 242 remain in contact with the implant 202 inside the tubular member 222 and exert an outward radial force, the proximal move of the hold-release structure 240 causes the implant 202 to proximally move with the hold-release structure 240 due to the friction between the implant 202 and the radially expandable members 242, allowing the implant 202 to be re-sheathed or recaptured into the tubular member 222 for repositioning.

With reference to FIGS. 3A-3C, the elongate tubular member 222 can be a sheath, microcatheter, or any other suitable tubular member defining a lumen. The elongate tubular member 222 may include a proximal end portion which may remain outside of the patient and is accessible to the user or physician when the endovascular system 200 is in use. The distal end portion of the tubular member 222 may be sized and dimensioned to reach a remote location in the vasculature of the patient, such as in a blood vessel adjacent to an aneurysm neck, a bifurcated blood vessel, an occlusion in a blood vessel, or the like. The hold-release structure 240 may be disposed at the distal end portion of the tubular member 222. While not shown, the elongate tubular member 222 may include one or more sections or regions each of which may have different configurations and/or characteristics. For example, the distal end portion of the elongate tubular member 222 may include a flexible section or region constructed e.g., of a coil to provide proper bending or deflection. A flexible distal end portion would allow the endovascular system 200 to navigate more easily through tortuous regions of the vasculature to remote locations in the patient. The proximal end portion may be constructed of a stiffer material e.g., of a rigid metal hypertube, to provide structural stability and sufficient pushability. In general, the elongate tubular member 222 or a section of the elongate tubular member 222 may be constructed from suitable metals such as stainless steel, nickel, titanium, nitinol, alloys of metals, biocompatible polymers, shape memory polymers, or combinations thereof. The distal end portion of the elongate tubular member 222 may have an outer diameter less than the outer diameter of the proximal end portion to reduce the profile of the distal end portion and facilitate navigation through tortuous vasculature. While not shown, the elongate tubular member 222 may include one or more markers which can be viewed e.g., via fluoroscopy, to assist the physician in operation of the endovascular system 200.

With reference to FIGS. 3A-3C the elongate delivery wire 224 has a proximal end portion and a distal end portion. The distal end portion of the delivery wire 224 couples the hold-release structure 240. The delivery wire 224 may include a distal tip 226 shaped or configured to make the delivery wire more flexible or atraumatic. While not shown, the distal end portion of the delivery wire 224 may include a section or region constructed e.g., of a coil to provide proper bending or deflection. One or more markers may also be coupled to the delivery wire to assist the physician to operate the endovascular system 200 via fluoroscopy. The delivery wire 224 may be constructed from suitable metals such as stainless steel, nickel, titanium, nitinol, alloys of metals, biocompatible polymers, shape memory polymers, or combinations thereof.

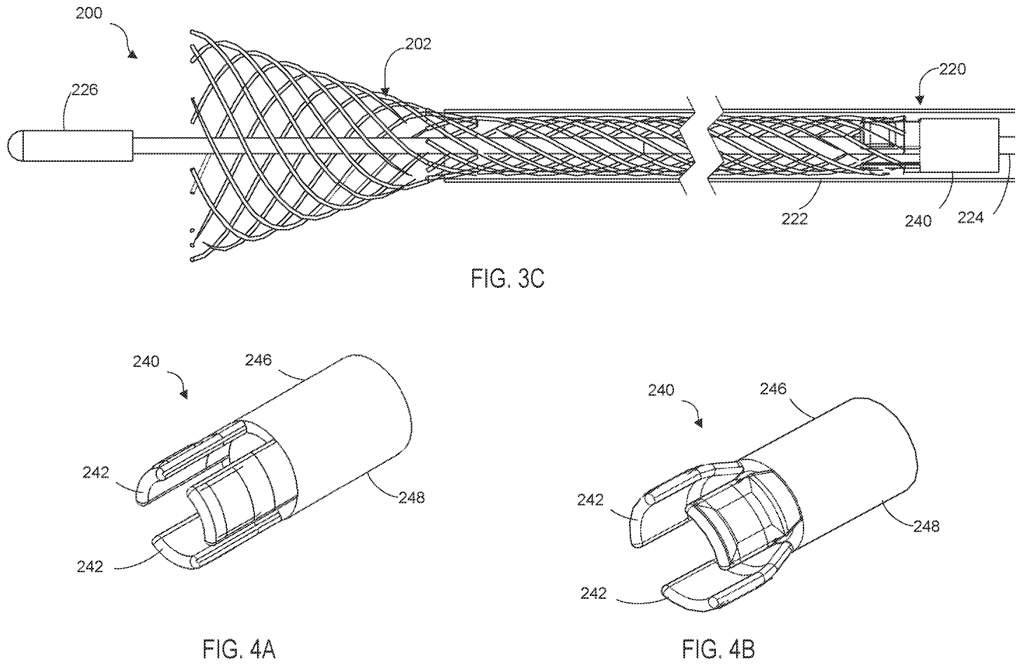

With reference to FIGS. 3A-3C and 4A-4B, the hold-release structure 240 may be coupled to the distal end portion of the delivery wire 224. The hold-release structure 240 can serve to hold or secure the implant 202 during delivery of the implant to the target site, re-sheath or recapture the implant 202 into the tubular member 222 for repositioning, and release or deploy the implant 202 to the target site.

With reference to FIGS. 3A-3C and 4A-4B, the hold-release structure 240 may be sized and/or shaped to be disposed within the tubular member 222. The hold-release structure 240 is slidably movable in the lumen of the tubular member 222. By way of example, the hold-release structure 240 may be moved distally relative to the tubular member 222 by pushing the delivery wire 224, and/or proximally relative to the tubular member 222 by pulling the delivery wire 224. Alternatively, the hold-release structure 240 may be moved relative to the tubular member 222, distally and/or proximally, by pulling and/or pushing the tubular member 222. The hold-release structure 240 may be coupled to the delivery wire 224 by any suitable means such as via soldering, welding, adhesive bonding, etc. In some embodiments, the hold-release structure 240 can be disposed within an expandable implant 202, which in turn can be disposed within the tubular member 222.

With reference to FIGS. 3A-3C and 4A-4B, the hold-release structure 240 may comprise two or more radially expandable members 242. The two or more radially expandable members 242 may have an expanded configuration when in a natural state and a collapsed configuration when compressed. In a preferred embodiment, the two or more radially expandable members 242 may be biased or formed such that expandable members 242 have a predetermined expanded configuration when in a natural state. When compressed or constrained, the two or more expandable members 242 can generate an outward radial force. Further, the two or more radially expandable members 242 may be configured to provide a sufficient friction force between the radially expandable members 242 and the implant 202. Therefore, when the implant 202 is loaded over the hold-release structure 240 within the tubular member 222, the outward radial force generated by the expandable members 242 allows the implant 202 to be held against the tubular member 222 for delivery. The friction force between the expandable members 242 and implant 202 allows the implant to be moved with the hold-release structure 240, proximally or distally, for deployment or recapturing of the implant 202. As used herein, the term “outward” and its grammatical equivalent refers to a direction or an orientation that is away from the delivery wire.

With reference to FIGS. 3A-3C and 4A-4B, the hold-release structure 240 can be constructed from a shape-memory material. Suitable shape-memory materials include nickel-titanium alloy Nitinol and other metal alloys. By way of example, the radially expandable members 242 can be formed by removing portions of a tubular member 246 of a shape-memory material. The wall of the tubular member 246 can be cut or shaped to form sections or expandable members biased to have a pre-determined expanded configuration when in a natural state. Any suitable techniques can be used to form the radially expandable members including laser cutting, etching, etc. known in the art. In a preferred embodiment, the radially expandable members 242 have an outer surface generally conforming to the inner surface of the implant 202 constrained within the tubular member 222 onto which an outward radial force may be exerted. In another embodiment, the outer surface of the radially expandable members may 242 be applied with a coating or a tacky pad to provide increased friction between the radially expandable members 242 and the implant 202.

With reference to FIGS. 4A-4B, in a preferred embodiment, the hold-release structure 240 comprises a proximal portion including a tubular body 248 and a distal portion including two or more radially expandable members 242 extending from the tubular body 248. The tubular body 248 may have an outer surface generally conforming to the inner surface of the implant 202 and/or the inner surface of the tubular member 222. The lumen defined by the tubular body 248 may have a size and/shape to receive the delivery wire 224. The tubular body 248 may be fixedly coupled to the delivery wire 224 via soldering, welding, adhesive bonding, etc. The radially expandable members 242 may be formed such that when constrained the two or more expandable members 242 constitute an extension of the tubular body 248 and provide an outer surface that generally conforming to the inner surface of the implant 202 within the tubular member 222.

With reference to FIGS. 3A-3C, the implant 202 can be any suitable implant compatible with the delivery system 200 of the disclosure. By way of example, the implant 202 can be an embolic device such as a stent, coil, or intrasaccular web used for treatment of brain aneurysms and/or peripheral thrombectomy. The implant 102 can also be a dilation device, filter, thrombectomy device, atherectomy device, flow restoration device, etc. for treatment of other disorders in other locations of a human body. For illustration purpose, the implant is shown in FIGS. 3A-3C as an expandable stent. It should be noted that the scope of the disclosure and appended claims are not limited to a specific type of an implant, and the hold-release structure disclosed herein can be used with any other suitable implants. In some embodiments, the outer surface of the implant 202 may be applied with a coating to reduce friction between the implant and tubular member 222 or to facilitate movement of the implant relative to the tubular member. In some embodiments, at least a portion of the inner surface of the implant 202 may be applied with a coating to provide increased friction between the implant and hold-release structure.

In operation, the implant 202 may be pre-loaded with the hold-release structure 240 inside a delivery sheath. The implant 202 and the hold-release structure 240 can be then transferred to a microcatheter to be delivered and deployed at a target site for treatment of a disorder within the vasculature of a patient. In an embodiment for treating neurovascular conditions such as aneurysm or for peripheral thrombectomy, a microcatheter may be introduced to the target site through an access e.g., in the femoral artery or groin area of the patient by using an introducer sheath or guiding catheter. The microcatheter may be guided to the target site through the use of a guidewire. The guidewire is visible via fluoroscopy, allowing the microcatheter to be reliably advanced over the guidewire to the target site.

Once the target site has been accessed with the microcatheter tip, the guidewire can be withdrawn, clearing the lumen of the microcatheter. The endovascular system 200 including the implant 202 and the delivery device 220 in a delivery configuration, can be placed into the proximal open end of the microcatheter and advanced through the microcatheter. When the implant 202 reaches the distal end of the microcatheter, it can be deployed from the microcatheter and positioned at the target site. The physician may advance and retract the implant 202 several times to obtain a desirable position of the implant within the vasculature. Once the implant 202 is satisfactorily positioned, the physician may push the delivery wire 224 distally, allowing the implant 202 to exit the delivery device, thereby releasing the implant 202 at the target site. The elongate tubular member 220 can be then removed from the microcatheter, and the microcatheter can be withdrawn from the vasculature of the patient.

Various embodiments of an endovascular system and a system for deploying implants within a human body have been described. Advantageously, the delivery system of the disclosure can enhance securement of the implant during delivery of the implant. The enhanced securement of the delivery system significantly reduces the risks of inadvertent or premature release of the implant when the delivery system is advanced or retracted in navigating through a tortuous vascular path in the human body.

Various embodiments of an endovascular system and a detachment system for deploying implants within a human body are described with reference to figures. It should be noted that the figures are intended to facilitate illustration and some figures are not necessarily drawn to scale. Further, in the figures and description, specific details may be set forth in order to provide a thorough understanding of the disclosure. It will be apparent to one of ordinary skill in the art that some of these specific details may not be employed to practice embodiments of the disclosure. In other instances, well known components or process steps may not be shown or described in detail in order to avoid unnecessarily obscuring embodiments of the disclosure.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The terms “coupled,” “supported,” “connected,” “mounted”, and variations are used broadly and encompass both direct and indirect couplings, supports, connections, and mounting. The term “proximal” and its grammatically equivalent refers to a position, direction or orientation towards the operator or physician's side. The term “distal” and its grammatically equivalent refers to a position, direction or orientation away from the operator or physician's side.

Those skilled in the art will appreciate that various other modifications may be made. All these or other variations and modifications are contemplated by the inventors and within the scope of the invention.

What is claimed is:

1. A system for delivering an implant in a patient, comprising:
a tubular member having a lumen;
a delivery wire having a proximal end portion and a distal end portion extending in the lumen of the tubular member; and
a hold-release structure coupled to the distal end portion of the delivery wire, the hold-release structure comprising two or more grasping members and being slidably movable in the lumen of the tubular member between a proximal first position and a distal second position, wherein in the proximal first position the two or more grasping members are constrained by the tubular member allowing the two or more grasping members to close and exert an inward clamping force to hold the implant, and wherein in the distal second position the two or more grasping members are unconstrained allowing the two or more grasping members to open to release the implant.

2. The system of claim 1, wherein the two or more grasping members comprise an inward step configured to contact an outer surface of the implant in exerting the clamping force.

3. The system of claim 2, wherein the hold-release structure is constructed from a material comprising a shape-memory material, and the two or more grasping members have a pre-determined open configuration when unconstrained.

4. The system of claim 4, wherein the hold-release structure comprises a tubular body and the two or more grasping members when constrained constitute an extension of the tubular body.

5. An endovascular system, comprising:
an implant; and
a delivery device operable to deploy the implant at a target site in a vasculature of a patient, wherein the delivery device comprises:
a tubular member having a lumen;
a delivery wire having a proximal end portion and a distal end portion extending in the lumen of the tubular member; and
a hold-release structure coupled to the distal end portion of the delivery wire, the hold-release structure comprising two or more grasping members and being slidably movable in the lumen of the tubular member between a proximal first position and a distal second position, wherein in the proximal first position the two or more grasping members are constrained by the tubular member allowing the two or more grasping members to close and exert an inward clamping force to hold the implant, and wherein in the distal second position the two or more grasping members are unconstrained allowing the two or more grasping members to open to release the implant.

6. The endovascular system of claim 5, wherein the implant comprises an embolic coil, a stent, or an intrasaccular web.

7. The endovascular system of claim 5, wherein the implant comprises a stent.

8. The endovascular system of claim 5, wherein the two or more grasping members comprise an inward step configured to contact an outer surface of the implant in exerting the clamping force.

9. The endovascular system of claim 8, wherein the hold-release structure is constructed from a material comprising a shape-memory material, and the two or more grasping members have a pre-determined open configuration when unconstrained.

10. The endovascular system of claim 8, wherein the hold-release structure comprises a tubular body and the two or more grasping members when constrained constitute an extension of the tubular body.

11. A system for delivering an implant in a patient, comprising:
a tubular member having a lumen;
a delivery wire having a proximal end portion and a distal end portion extending in the lumen of the tubular member; and
a hold-release structure coupled to the distal end portion of the delivery wire, the hold-release structure comprising two or more radially expandable members and being slidably movable in the lumen of the tubular member between a proximal first position and a distal second position, wherein the two or more radially expandable members are configured to exert an outward radial force when constrained by the tubular member allowing the hold-release structure to hold an implant against the tubular member in the proximal first position, and wherein the two or more radially expandable members are configured to create a friction force on the implant allowing the hold-release structure to move the implant relative to the tubular member from the proximal first position to the distal second position.

12. The system of claim 11, wherein the two or more radially expandable members are configured to exert the outward radial force to an inner surface of the implant.

13. The system of claim 12, wherein the hold-release structure is constructed from a shape-memory material forming the two or more radially expandable members in a pre-determined open configuration when unconstrained.

14. The system of claim 12, wherein the two or more radially expandable members comprise a contact surface having a shape generally conforming to the inner surface of the implant.

15. The system of claim 12, wherein the two or more radially expandable members comprise a coating or pad configured to provide an increased friction force between the two or more radially expandable members and the implant.

16. An endovascular system, comprising:
an implant; and
a delivery device operable to deploy the implant at a target site in a vasculature of a patient, wherein the delivery device comprises:
a tubular member having a lumen;
a delivery wire having a proximal end portion and a distal end portion extending in the lumen of the tubular member; and
a hold-release structure coupled to the distal end portion of the delivery wire, the hold-release structure comprising two or more radially expandable members and being slidably movable in the lumen of the tubular member between a proximal first position and a distal second position, wherein the two or more radially expandable members are configured to exert an outward radial force when constrained by the tubular member allowing the hold-release structure to hold the implant against the tubular member in the proximal first position, and wherein the two or more radially expandable members are configured to create a friction force on the implant allowing the hold-release structure to move the implant relative to the tubular member from the proximal first position to the distal second position.

17. The endovascular system of claim 16, wherein the implant comprises an embolic coil, a stent, or an intrasaccular web.

18. The endovascular system of claim 16, wherein the implant comprises a stent.

19. The endovascular system of claim 18, wherein the two or more radially expandable members are configured to exert the outward radial force to an inner surface of the stent.

20. The endovascular system of claim 16, wherein the hold-release structure is constructed from a shape-memory material forming the two or more radially expandable members in a pre-determined open configuration when unconstrained.

21. The system of claim 20, wherein the two or more radially expandable members comprise a contact surface having a shape generally conforming to the inner surface of the implant.

22. The endovascular system of claim 21, wherein the two or more radially expandable members comprise a coating or pad configured to provide an increased friction force between the two or more radially expandable members and the implant.
